(19) United States
(12) Patent Application Publication
(10) Pub. No.: US 2014/0314872 A1
(43) Pub. Date: Oct. 23, 2014

(54) Methods of Use of Culture Supernatant Obtained from Mesenchymal Stem Cells from Dogs and Cats for Treatment of Organ Dysfunction

(71) Applicant: Hans Klingemann, Boston, MA (US)

(72) Inventor: Hans Klingemann, Boston, MA (US)

(21) Appl. No.: 14/258,761

(22) Filed: Apr. 22, 2014

Related U.S. Application Data
(60) Provisional application No. 61/854,241, filed on Apr. 22, 2013.

(51) Int. Cl. A61K 35/28; A61K 38/19; C12N 5/0775

(52) U.S. Cl. CPC A61K 35/28; C12N 5/0662; A61K 38/19
USPC 424/572

(57) ABSTRACT

A method of treatment of organ dysfunction using a supernatant containing molecules, cytokines and vesicles secreted from mesenchymal stem cells (MSC) from animals is provided. The MSC are cultured, and then centrifuged to separate them from the desired supernatant containing the molecules and vesicles that may be used for treatment. Treatment may be for dogs, cats, and similar animals. This treatment may be for any number of ailments including, but not limited to mitigation or reversal of complications from heart, kidney, liver, or other organ impairment.

### CROSS-REFERENCE TO RELATED APPLICATIONS

This application is a non-provisional application which claims the benefit to Provisional Application No. 61/854,241 filed on Apr. 22, 2013.

### BACKGROUND OF THE INVENTION

1. Field of the Invention

The present invention relates generally to biological therapies, namely a treatment method for animals using stem cells. More specifically, the present invention relates to a method of treatment of animal organ dysfunction using a supernatant obtained from mesenchymal stem cells.

2. Description of Related Art

Stem cells, especially mesenchymal stem cells (MSC) are considered potentially useful for the treatment of a large variety of canine and feline conditions and disorders, for example, but not restricted to the replacement and repair of tissues such as cartilage, bone, pancreatic islets, severed nerve cells, skin grafts for burns or abrasions, and treatment of organ failure and dysfunction such as kidney, heart and liver.

It is increasingly being recognized that MSC produce growth factors and cytokines, and a number of other peptides and proteins collectively named the “Secretome”. These molecules are secreted by MSC into the supernatant of the culture either with or without stimulation. Simulation may be actions such as contact with tissue or/and activating molecules. The MSC also release vesicles which likewise contain a number of bioactive molecules. Other terms for vesicles are microvesicles, exosomes or microsomes. Some of the beneficial effects seen with the infusion of MSC in humans are being attributed to the paracrine effect of MSCs (i.e. the production and release of those bioactive molecules and vesicles) not necessarily requiring direct contact of MSC with the target tissue.

Examples for such a paracrine effect in humans are improvement of kidney, liver or heart function after infusions with MSC. In several cases those infused MSC could not be found in the affected organ strongly suggesting a paracrine effect. Therefore, what is needed is a treatment method that may utilize these secreted molecules and vesicles from stem cells such as MSC without treating with the entire cell.

### SUMMARY OF THE INVENTION

The subject matter of this application may involve, in some cases, interrelated products, alternative solutions to a particular problem, and/or a plurality of different uses of a single system or article.

In one aspect, a method of treatment of organ dysfunction using a supernatant containing molecules and vesicles produced by and secreted from mesenchymal stem cells (MSC) from animals is provided. Initially, a quantity of MSC are obtained. These cells are allowed to culture (grow and multiply) in a medium containing a serum or standard medium, and then again transferred into a serum free medium. The cells may be washed before transfer to remove any traces of serum. Once the cells are cultured in the serum free medium for various length of time, they may be centrifuged to separate the MSC from the supernatant. The remaining supernatant comprises the molecules and vesicles secreted from the MSC (“Secretome”), but not the MSC themselves. This supernatant may be gathered, and used for treatment purposes. The collected supernatant may or may not be further processed to enrich for specific molecules, exosomes, microvesicles or other vesicles. Treatment may be for dogs, cats, and similar animals. This treatment may be for any number of ailments including, but not limited to mitigation or reversal of complications from heart, kidney, liver, or other organ impairment.

### BRIEF DESCRIPTION OF THE DRAWINGS

FIG. 1 provides a flow chart of one embodiment of the steps of the present invention.

FIG. 2 provides a flow chart of another embodiment of the steps of the present invention.

FIG. 3 provides a flow chart of yet another embodiment of the steps of the present invention.

### DETAILED DESCRIPTION

The detailed description set forth below in connection with the appended drawings is intended as a description of presently preferred embodiments of the invention and does not represent the only forms in which the present invention may be constructed and/or utilized. The description sets forth the functions and the sequence of steps for constructing and operating the invention in connection with the illustrated embodiments.

Generally, the present invention concerns a method of treatment of animals such as dogs and cats using mesenchymal stem cells (MSC). Particularly, the method involves a use of molecules and vesicles (including microvesicles, exosomes, and the like) that are secreted from the MSC into a supernatant. Typically, the treatment composition is separated from the MSC and thus contains no MSC. Generally, the method includes the steps of extracting and isolating these molecules and/or vesicles into one or a plurality of solutions or suspensions. Further the method includes use of these solutions or suspensions in a treatment method for the animals. The treatment method may include: systemic injection, infusion, direct injection into an area of treatment, topical and/or localized application, and the like. Preferably, the treatment involves injection of the solutions containing secreted molecules and vesicles isolated from the MSC. As such, the method involves at least two phases: the first phase being a preparation phase for gathering and isolation of molecules and vesicles secreted from the MSC into a supernatant, the second phase being for treatment using the supernatant containing molecules and vesicles. This supernatant may then be used for treatment, or processed further. The term vesicle will be used herein as a general term to include microvesicles, exosomes, microsomes and the like.

The preparation phase of the methods contemplated herein begins with isolated MSC from an animal such as a dog or cat. These MSC may be obtained from any tissue including, but not limited to, fats such as adipose tissue, bone or bone marrow, umbilical cord, gonads (such as testicles and ovaries), and the like. In one embodiment, the MSC may be obtained from canine or feline testes and/or ovaries as taught in U.S. patent application Ser. No. 13/815,422 entitled Isolation and Methods of Use for Mesenchymal Stem Cells Obtained from Canine and Feline Testes and Ovaries, incorporated herein by reference. A purity of the MSC may be confirmed, and then the cells are expanded in a culture. In one embodiment, the MSC may be expanded to confluence. This expansion step may be performed using a medium such as a medium containing serum such as fetal bovine serum (FBS). In a particular embodiment, the MSC may be expanded in standard culture conditions using a minimal essential medium such as DMEM along with a serum such as 20% FBS. In one embodiment, the MSC may be allowed to expand in this culture state for 24-48 hours. In another embodiment, the cell cultures may go on for some time, with a medium split every few days. The MSC may be obtained from the animal intended to be treated in one embodiment, or may be obtained from an animal of the same species. In another embodiment, a number of different animals (of the same species) may provide the MSC, and these MSC may be pooled to create a generic therapeutic as the final supernatant result. The MSC may also be pooled with MSC from different tissue sources from the same animal, or different animals of the same species.

Once the cells have expanded adequately, they may be shifted to a serum free medium for additional culturing. One example of a serum free medium may be Mesencult basal medium from Stem Cell Technologies, Vancouver, BC, Canada, X-vivo from Lonza or DMEM based medium. The cells are again allowed to culture in this medium for a period. In one embodiment, the culture period is 24-48 hours. In another embodiment, the cells may be allowed to culture to confluence. It should be understood that the second serum free culturing phase may vary in length and extent, and may go longer or shorter than the approximate culture period of 24-48 hours, for example, the cells may be allowed to culture in the serum free medium for approximately six hours in another embodiment.

In some embodiments, the MSC may be cultured in a serum free medium for the entire culturing process.

After the first culture step, the MSC may be washed with a phosphate buffer saline (PBS), or similar solution, to remove any residues of FBS that may be attached to the cells. It is important that the medium which will ultimately provide the liquid phase supernatant be serum free because the serum irrespective of its origin (serum or human) contains vesicles, proteins, RNA, and the like and may interfere with the therapeutic benefit of the supernatant

In yet another embodiment, the serum or medium containing culture supernatant may be centrifuged, and the resulting pellet of cells may be re-suspended, washed, and placed into the second culture with serum free medium. After, for example, 12-48 hours of culture, the supernatant can then be used as is without further preparation, or it can be further processed. In the case of further processing, the supernatant is removed and centrifuged again (multiple times—ultracentrifugation) and even possibly put over a sucrose supernatant. That final supernatant after all those steps is enriched for bioactive molecules, exosomes vesicles and the like, and can also be used for therapeutic purposes. It should be understood that any of those resulting solutions, herein referred to as supernatants, can be frozen for later use.

In many embodiments, the cells may be washed between their transfer from the serum based medium to the serum free medium. This may achieve a number of goals, namely removing all exosomes and vesicles present from the serum. In further embodiments, various centrifugation and/or ultracentrifugation steps may occur, with various washings of the pellet between some or all of the steps. In still another embodiment, after the initial transfer of the MSC from the serum based medium to the serum free condition, and after allowing the vesicles from the MSC to diffuse into the serum free supernatant, the supernatant may then undergo one or a number of centrifugation steps with one or repeated washings. In some aspects of this embodiment, the pellet may be removed after one centrifugation step and before another centrifugation step.

During the culturing steps the MSC are secreting a number of molecules, chemicals, cytokines, exosomes and vesicles (here called collectively “bioactive molecules”. These “bioactive molecules” are the desirable components for the methods herein. To isolate them further from the MSC, a centrifuging process may be used. This causes the MSC to move to a bottom of a centrifuge tube forming a pellet, leaving the bioactive molecules in a supernatant fluid above the pellet. In some embodiments, the MSC pellet may be discarded.

In one embodiment, the centrifugation process may be performed for 5-30 minutes at 300-10,000 g or even higher up to 150,000 g in some embodiments after the MSC have been centrifuged into the pellet and have been removed. This higher speed centrifugation will sediment the bioactive molecules, vesicles or exosomes that can then be used for therapeutic purposes. Instead of centrifugation, various other methods may be applied to separate out the bioactive molecules, vesicles and exosomes from the supernatant.

In one embodiment, the collected supernatant fluid may be concentrated.

In another embodiment, the collected supernatant fluid may be cryopreserved. In one embodiment, this cryopreservation may be performed using liquid nitrogen.

In a further embodiment, the collected supernatant may be further processed to isolate different components from the supernatant, namely bioactive molecules, microvesicles and exosomes. This may be performed in multiple centrifugation steps, or through commercial separation kits and processes.

In some embodiments, centrifugation may go on for many hours, even overnight or longer. This may be performed at approximately 20 C. Any suspended vesicles will be drawn to a bottom of a centrifuged tube, forming a pellet. The secondary supernatant containing only the molecules secreted from the original MSC may then be collected, concentrated, and/or cryopreserved. Moreover, the vesicle pellet may be re-suspended in PBS or dissolved in lysis buffer, and can be used for therapy directly, or added to other supernatant solutions to enhance their efficacy.

Further processes for removal of the vesicles for creating the secondary supernatant include high speed centrifugation (for example, 100,000 g for one hour). This creates a pellet that contains vesicles including micro vesicles and exosomes. Those can be further processed by performing centrifugal filtration through, for example, a 0/22 micron filter (such as for 2 minutes at 750 g). Larger micro vesicles are recovered from a top of the filter by washing, and the flow through would contain smaller exosomes.

In another embodiment, vesicles such as exosomes from the supernatant can be further enriched by sucrose gradient where the sucrose is layered on top of the exosomes suspension in a tube. The sample is then centrifuged at 100,000 overnight at 4° C. Gradient fractions are collected from the top of the tube, diluted with PBS, and again ultracentrifuged at about 100,000 for 1 h. Exosomes in each fraction can be used without further treatment of they can be lysed and suspended in PBS or medium.

Once isolated, the collected supernatant, secondary supernatant, MSC pellet, and/or vesicle/exosome pellet may be combined in any manner for treatment by injection/infusion, topical application, and the like, or may be used for treatment on their own. For example, a quantity the initial collected supernatant may have the vesicle pellet added from another quantity of secondary supernatant, thereby adding additional vesicles to the mixture. In other embodiments, this mixing may be performed in any manner to achieve a final injectable treatment mixture. For the purposes of further discussion, the term “supernatant” may be used to refer to not only the processed supernatant, but any mixture containing the supernatant, secondary supernatant, vesicles and/or MSC. Preferred supernatant compositions include a secondary supernatant containing only the secreted molecules from the MSC; a supernatant containing the secreted molecules and vesicles; and a supernatant containing the secreted molecules and vesicles, along with added vesicles separated from another supernatant solution.

Once the supernatant treatment mixture containing the desired components is determined, the treatment phase may begin. Treatment may involve any method of getting the MSC supernatant and contents to an area requiring treatment. While the mesenchymal stem cell based supernatants may be used for many therapeutic and treatment purposes, the present invention focuses largely on treatment of organ dysfunction, though the present invention is in no way limited to this particular treatment.

In organ dysfunction treatments, the treatment phase involves getting the supernatant solution to the particular organ requiring treatment. For example, for treatment of liver dysfunction, the liver is the intended destination for the supernatant solution. As such, liver treatment may be performed in any number of ways, including, but not limited to systemic injection such as an intravenous injection, subcutaneous injection, intramuscular injection, and the like.

Another option for introduction of the supernatant solution to the targeted area is a direct injection into the organ or area requiring treatment. In some direct injection embodiments, the supernatant solution may further comprise a compound configured to keep the solution in the area to be treated, and preventing its dissipation. For example, a vasoconstrictor such as epinephrine, norepinephrine, vasopressin, phenylephrine, pseudoephedrine, and the like.

In further embodiments, supernatants of different compositions may be injected for treatment at different phases. For example, a supernatant containing only secreted molecules may be initially injected, followed by a supernatant containing a saline solution (or similar) with suspended vesicles from the MSC only. These different compositions may further be applied in different injection methods, for example the supernatant containing secreted molecules may be applied by a direct injection to the area to be treated, while the solution with suspended vesicles may be intravenously injected. It should be understood that any variation of supernatant may be provided in different combinations and treated in the different manners as described. Moreover, in some embodiments, a topical application of the MSC supernatant may be used to treat disorders that involve the skin.

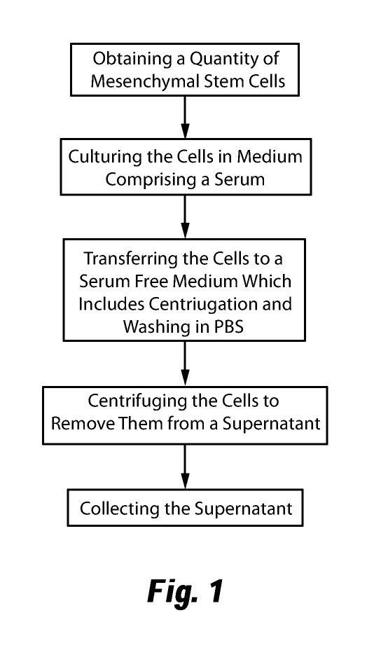

Turning now to FIG. 1 a flow chart of one embodiment is provided. The flow chart relates to the preparation of the supernatant. The method begins with the step of obtaining a quantity of mesenchymal stem cells. These stem cells may be obtained, as noted above, from a number of sources. Once the cells are obtained and confirmed, they may be cultured in a medium comprising a serum. This serum may be Human, Bovine, Horse, or other serum. Once the cells have cultured adequately in the serum based medium, they may be transferred to a serum free medium for further culturing. It may be important to culture the cells in a serum free medium for at least a period because serum based medium have vesicles, molecules, and the like, that will contaminate the composition of the supernatant collected form MSC

Once the cells have been cultured in the serum free medium for a time sufficient for them to secrete vesicles and bioactive molecules from the MSC, the cell culture may be centrifuged, thereby separating the cells from a supernatant. The supernatant will comprise the serum free medium, along with the secreted vesicles and bioactive molecules. This supernatant may then be collected and used for treatment and/or stored.

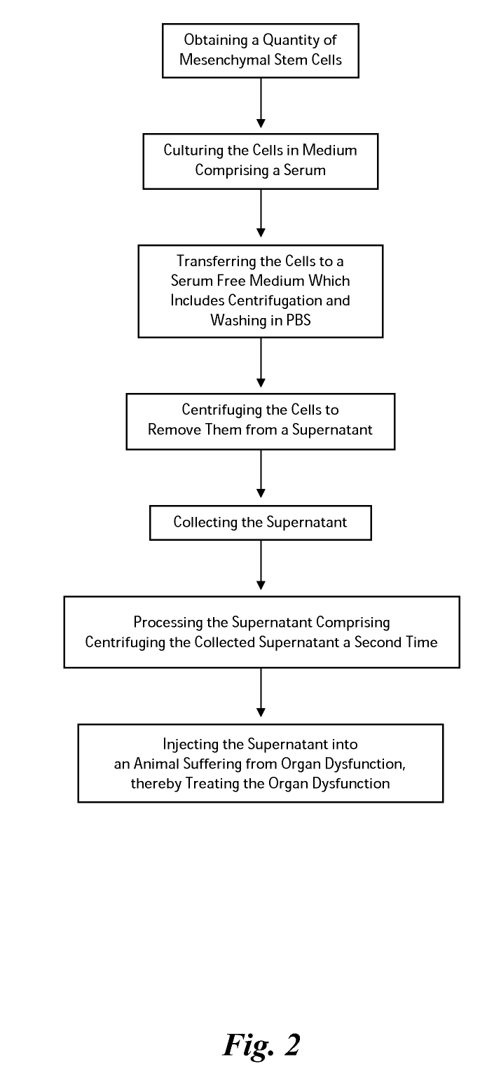

FIG. 2 shows a flow chart of another embodiment of the present invention. The flow chart involves the preparation of the supernatant steps shown in FIG. 1, along with a number of further steps. Once the supernatant has been prepared and collected, for example as noted in FIG. 1, the supernatant may be further processed into a secondary supernatant. The secondary supernatant may be free of vesicles, and contain only molecules such as proteins secreted from the MSC. In one embodiment, the secondary supernatant is prepared by further centrifugation, causing the larger vesicles to leave the secondary supernatant. Once the secondary supernatant has been prepared, it, or the original supernatant may be used for treatment by, for example, injecting it into an animal suffering from organ dysfunction.

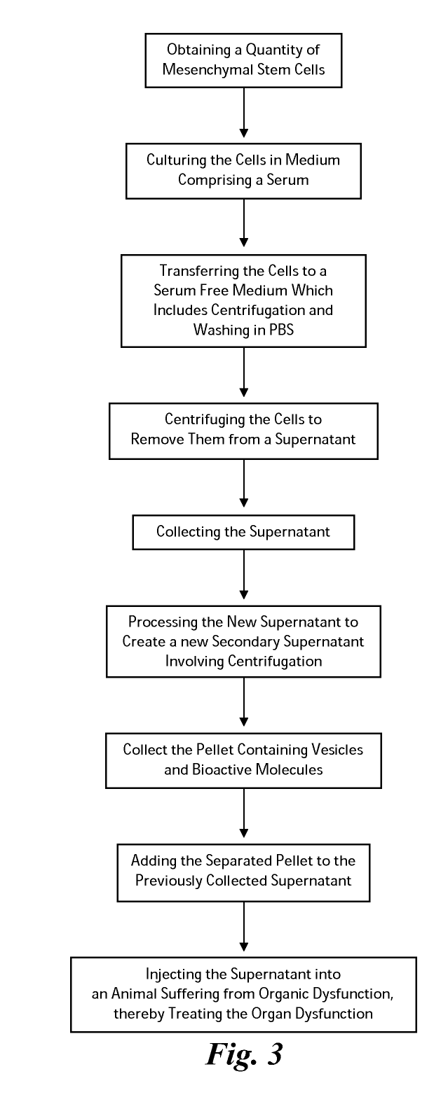

FIG. 3 provides a view of another embodiment of the present invention. The method begins with the step of obtaining a quantity of Mesenchymal stem cells. These cells are cultured in a medium comprising a serum. Next, the cells may be centrifuged, washed with a PBS solution, and then transferred to a serum free medium. The cells are again allowed to culture for a determined amount of time. Once cultured, the cells are centrifuged to remove them from a supernatant medium they were cultured in. In this embodiment, the supernatant is further processed to create a secondary supernatant. The secondary supernatant processing begins with again centrifuging the supernatant, preferably at a higher g or for longer than before. This second centrifuging step serves to remove larger vesicles from suspension. The secondary supernatant having the vesicles removed may then be collected. In further embodiments, the separated vesicles may be removed in the form of a pellet, and then may be added to a previously collected supernatant, to enrich the supernatant, or may be re-suspended on their own in the supernatant, medium or a PBS solution. Finally, the enriched supernatant, and/or the secondary supernatant may be injected into an animal suffering from organ dysfunction as a form of treatment.

While several variations of the present invention have been illustrated by way of example in preferred or particular embodiments, it is apparent that further embodiments could be developed within the spirit and scope of the present invention, or the inventive concept thereof. However, it is to be expressly understood that such modifications and adaptations are within the spirit and scope of the present invention, and are inclusive, but not limited to the following appended claims as set forth.

What is claimed is:

1. A method of treatment of organ dysfunction using mesenchymal stem cells from animals comprising the steps of:
obtaining a quantity of mesenchymal stem cells;
preparing a supernatant solution, the preparing comprising:
culturing the MSC in medium comprising a serum or plasma;
washing and transferring the cells to a serum free medium after the culturing for a second culturing step;
centrifuging the cells to remove them from a supernatant after the second culturing step is completed, the supernatant containing molecules, chemicals, cytokines and vesicles (“bioactive molecules”) secreted from the cells; and
collecting the supernatant;
administering the supernatant into an animal suffering from organ dysfunction, thereby treating the organ dysfunction.

2. The method of claim 1 further comprising the step of processing the supernatant into a secondary supernatant, the step of processing the supernatant comprising centrifuging the collected supernatant a second time, the centrifuging selected to separate the bioactive molecules and vesicles from the supernatant, leaving only the molecules in the supernatant.

3. The method of claim 1 wherein the step of centrifuging the cells comprises centrifuging for 5-30 minutes at 300-10,000 g.

4. The method of claim 2 wherein the step of centrifuging the collected supernatant a second time comprises centrifuging for approximately 60 minutes at approximately 100,000 g.

5. The method of claim 1 further comprising the step of processing the supernatant into a secondary supernatant, using a commercial separation kit.

6. The method of claim one where the pellet after centrifugation is re-suspended in medium or PBS and either added to the first supernatant or used as is.

7. The method of claim 1 further comprising the step of cryopreserving the collected supernatant or the resuspended pellet.

8. The method of claim 1 further comprising the step of adding a second quantity of vesicles to the collected supernatant.

9. The method of claim 1 further comprising the step of concentrating the collected supernatant.

10. The method of claim 1 wherein the step of administering comprises an intravenous injection.

11. The method of claim 1 wherein the step of administering comprises an injection directly into or adjacent to an organ or tissue pathology to be treated.

12. The method of claim 1 wherein the step of administering comprises topical administration

13. The method of claim 1 further comprising the steps of:
recovering a pellet of the cells after the centrifuging step;
re-culturing the recovered cells, the re-culturing comprising:
culturing the cells in medium comprising a second serum;
transferring the cells to a second serum free medium after the culturing for a further culturing step;
washing the cells after the further culturing step with the phosphate buffer saline;
re-centrifuging the re-cultured cells to obtain a new supernatant; and
injecting the new supernatant into the animal suffering from organ dysfunction, thereby treating the organ dysfunction.

14. The method of claim 2 wherein the separated bioactive molecules and vesicles are separated from the supernatant in the form of a pellet, and further comprising the step of re-suspending the pellet in a solution.

15. The method of claim 12 wherein the step of injecting the second supernatant is performed at a different injection site than the injection of the supernatant.

16. The method of claim 1 wherein the mesenchymal stem cells are obtained from the animal to be treated.

17. The method of claim 1 wherein the mesenchymal stem cells are obtained from an animal different from the animal to be treated but of the same species.

18. The method of claim 2 further comprising the step of cryopreserving the supernatant.

19. The method of claim 1 wherein the step of transferring the cells to a serum free medium for a second culturing step comprises the steps of:
washing the cells before transferring the cells; and
culturing the cells in the serum free medium for 24-48 hours.

20. The method of claim 1 wherein the mesenchymal stem cells are not injected and the step of administering comprises injecting only the collected supernatant being free of the mesenchymal stem cells.

21. A method of mesenchymal stem cell treatment for a feline or canine comprising the steps of:
obtaining a quantity of mesenchymal stem cells;
preparing a supernatant solution, the preparing comprising:
culturing the cells in medium comprising a serum;
washing the cells and transferring the cells to a serum free medium after the culturing for a second culturing step; centrifuging the cells to remove them from a supernatant, the supernatant containing bioactive molecules and vesicles secreted from the cells; and
collecting the supernatant;
injecting only the supernatant into an animal suffering from the disorder, thereby treating the disorder.
